(19) United States
(12) Patent Application Publication
(10) Pub. No.: US 2007/0209950 A1
(43) Pub. Date: Sep. 13, 2007

(54) METHODS AND DEVICES FOR THE DETECTION AND MEASUREMENT OF FREE METALS IN FLUIDS AND METHODS FOR DIAGNOSING METAL-RELATED DISEASES AND FOR DETERMINING PHARMACOLOGIC DOSING REGIMENS

(76) Inventors: John S. ALTHAUS, Ann Arbor, MI (US); Steve H. KANZER, Ann Arbor, MI (US)

Correspondence Address: HAYES SOLOWAY P.C., 3450 E. SUNRISE DRIVE, SUITE 140, TUCSON, AZ, 85718, US

(21) Appl. No.: 11/674,036

(22) Filed: Feb. 12, 2007

Related U.S. Application Data
(60) Provisional application No. 60/771,979, filed on Feb. 10, 2006.

(51) Int. Cl. G01F 1/64; G01N 33/487

(52) U.S. Cl. 205/792; 204/403.01

(57) ABSTRACT

A detection method or a method for a point of care diagnostic assay which comprises detecting non-covalently bound copper (free copper) concentrations in a fluid.

### CROSS REFERENCE TO RELATED APPLICATION

This application claims priority from U.S. Provisional Application Ser. No. 60/771,979, filed Feb. 10, 2006 the contents of which are incorporated herein by reference.

BACKGROUND OF THE INVENTION

I. Field of Invention

The present invention relates to methods and devices for detecting the presence and concentration of metals and metal compounds in fluids. The invention has particular utility for detecting metal levels, such as copper or iron, in plasma and serum in animals, including humans, and in diagnosing metal-related diseases in humans and animals, and will be described in connection with such utilities, although other utilities are contemplated.

II. Description of the Related Art

A number of diseases and health conditions have been linked to metal levels in the body. In some cases, diseases are associated with elevated levels while in others lowered serum values. Convenient direct measurement of free and bound copper and iron in body fluids is a significant medical need. Still further, serum copper can exit in discreet yet separate pools. Though total copper may not differ, distribution within pools may vary substantially between samples. Measurement of copper and iron provides functions that are diagnostic, prognosticative and/or maintenance-serving.

Prior to the present invention, currently available methods of estimating free and bound copper and iron in plasma and serum rely upon estimations that are subject to a considerable amount of variability and inaccuracy. For example, the current “gold standard” method for detecting “free serum copper” levels involves a measurement of total serum copper (generally determined by flame atomic absorption spectroscopy) and subtracting the estimated amount of copper theoretically bound to the serum protein ceruloplasmin. Such estimation can be highly inaccurate due to the variation in actual copper-ceruloplasmin binding that varies between individuals as well as factors such as aging during a person's lifetime. For example, it is widely assumed that a single ceruloplasmin protein binds 6-7 copper atoms. However, with aging the binding capacity of ceruloplasmin can decrease to 5 copper atoms. Since an elevated pool of free copper in serum can be toxic to the central nervous system and other organ systems, what is needed, and the present invention provides, is an instrument and methodology capable of directly measuring free and bound copper without relying on estimations that may or may not be correct. A preferred embodiment of the present invention also provides a method of diagnosing persons having potentially toxic elevated free copper pools as well as means of dosing one or more copper lowering agents based upon direct measurements of free serum copper pools. Examples of diseases that may be associated with elevated free serum copper pools include: hepatolenticurlar degeneration (Wilson's disease), Alzheimer's disease, Parkinson's disease, schizophrenia, atherosclerosis, diabetes mellitus, and other common diseases.

Copper Related Diseases:

Ceruloplasmin, like ferritin, is an acute-phase reactant. Consequently, serum copper and ceruloplasmin levels are increased in several conditions including any inflammatory process, pregnancy, coronary artery disease, diabetes, malignancies, and renal failure. It should be noted that in the elderly, two conditions can lead to elevated copper in the brain. First, the blood brain barrier become more permeable as we age and thereby allows exchange between the serum and the CSF to occur more easily. Secondly, as liver function diminishes, the amount of copper associated with ceruloplasmin decreases, from 6-7 copper atoms per ceruloplasmin molecule to 5 copper atoms per ceruloplasmin molecule making free copper more available for transport to the brain. Other causes of increased copper levels include accidental consumption by children, contaminated water sources, suicide attempts, and topical creams for bum treatment that contain copper salts. Copper toxicity has been shown to be associated with the following signs, symptoms, and disease states: jaundice, nausea, vomiting, epigastric pain, headache, syncope, hypotension, generalized weakness, diarrhea, hemochromatosis, hemolytic anemia, oliguria, hemoglobinuria, hepatic necrosis, renal failure, coma. Conversely, some signs, symptoms and disease states associated with copper deficiency are: loss of color in the hair and skin, baldness, anemia, leucopenia, neutropenia, inflammation, skin sores, cardiovascular damage, elevated low density lipoprotein (LDL) cholesterol levels, benign prostatic hyperplasia, brain disturbances, diarrhea, generalized weakness, hypoglycemia, impaired immune function, impaired respiratory function, bone and joint abnormalities, arthritis, connective tissue weakening, osteoporosis, pathologic fractures, and retinal degeneration. Both serum levels of copper and ceruloplasmin are often low in copper deficiency. Copper deficiency has been reported in malnourished premature infants, patients with chronic diarrhea, patients with remote history of gastrectomy including Roux-En-Y gastric bypass surgery for morbid obesity, patients on chronic peritoneal dialysis, and in patients receiving prolonged parenteral nutrition without any appropriate copper supplementation.

Wilson's Disease:

Wilson disease is an autosomal recessive inherited disorder of copper metabolism. The condition is characterized by excessive copper deposition in the liver, brain, cornea, kidneys, and other tissues. The major physiologic aberration is decreased transport of copper from the liver into bile, with concomitant decreased biliary excretion of copper. The genetic defect, localized to the long arm of chromosome 13, has been shown to affect the copper-transporting adenosine triphosphatase (ATPase) gene (ATP7B) in the liver. Patients with Wilson disease usually present with liver disease during the first decade of life subsequent with neurologic illness during the second and third decade. The diagnosis is confirmed by measurement of serum ceruloplasmin, 24 hour urinary copper excretion, serum free copper and hepatic copper content, as well as the slit lamp detection of Kayser-Fleischer rings (copper deposits in the cornea of the eye) and imaging of the brain.

Unfortunately, most patients with Wilson's disease are diagnosed after liver cirrhosis or neurologic damage already developed. Although liver transplantation is an available therapeutic modality, many of these patients suffer to die or live with severe neurologic sequelea. However, if the diagnosis is made at an early stage of the clinical course, they can have a normal life back by starting an early treatment with an appropriately dosed orally applied chelating agent, e.g. penicillamine, without any further complications. Therefore early diagnosis and treatment in Wilson's disease is very important.

Menkes's Disease:

Menke's Disease is an X-linked genetic disorder that results in severe progressive neurodegeneration caused by a genetic defect in the transport of copper from the intestine, leading to low serum copper and ceruloplasmin concentrations. Because it is carried on the X chromosome, the disease primarily affects male infants. Copper accumulates at abnormally low levels in the liver and brain, but at higher than normal levels in the kidney and intestinal lining. Affected infants may be born prematurely. Symptoms typically appear during infancy with normal or slightly slowed development proceeding for 2 to 3 months, followed by severe developmental delay and loss of early developmental skills. Menke's Disease is also characterized by seizures, hypotonia, failure to thrive, temperature instability, hypoglycemia, osteoporosis, and strikingly peculiar hair, described as kinky, colorless or steel-colored, and easily broken. There can be extensive neurodegeneration in the gray matter of the brain. Arteries in the brain can also be twisted with frayed and split inner walls leading to rupture or blockage. Most patients do not survive past 3 years of age.

Alzheimer's Disease:

The relation between serum free copper and Alzheimer's Disease has received considerable attention over the last few years. Copper is an essential element and under normal physiologic circumstances is maintained complexed to proteins. This appears to be a protective mechanism developed in mammals to prevent “free copper” availability. In the free form copper is a highly reactive metal causing free radical formation and catalyzing oxidative damage. Normally, most copper is bound to ceruloplasmin in the serum. In the brain, protective mechanisms have also evolved to keep copper complexed to intracellular proteins and those in the cerebral spinal fluid (CSF). Copper is shown to be bound to the Tau protein, amyloid precursor protein, beta amyloid protein, and apoE protein. We believe copper is normally bound to these proteins as a protective mechanism against excess copper. Indeed, in vitro studies have shown that amyloid precursor protein expression is down regulated in copper depleted cells. The presence of beta amyloid plaques and intracellular neurofibullary tangles appear to result from copper binding to beta amyloid and the tau protein, respectively, and induction of protein structural changes. This appears to be a pathological mechanism in response to excess free copper. Thus, under conditions of excess free copper in the brain, these protein structural changes appear to be the markers of the disease and not the cause. Interestingly, the work of Squitti and associates. Neurology. 2005 Mar. 22;64(6): 1040-6., have shown that the level of copper unassociated with ceruloplasmin is markedly elevated in Alzheimer's Disease subjects compared to age-matched controls.

In addition, the work of Sparks et al. Proc Natl Acad Sci U S A. 2003 Sep. 16;100(19):11065-9 have shown a direct association between copper in the drinking water in an experimental rabbit model of cholesterol-diet induced Alzheimer's Disease. In this work these investigators demonstrated that animals consuming distilled water had markedly reduced Alzheimer's Disease plaques in the brain compared to tap water controls. Furthermore, they determined that copper was the culprit mineral in the water that induced this effect. The cholesterol-diets likely caused endothelial damage to the blood brain barrier allowing the easy penetration to the brain compartment. The tap water drinking rabbits also suffered dramatically poorer performance in complex tests of memory.

Amyotrphic Lateral Sclerosis (ALS):

In addition, the work of Goto JJ, et al. J Biol Chem. 2000 Jan. 14;275(2):1007-14 teach a loss of in vitro metal ion binding specificity in mutant copper-zinc superoxide dismutases associated with familial amyotrophic lateral sclerosis. The presence of the copper ion at the active site of human wild type copper-zinc superoxide dismutase (CuZnSOD) is essential to its ability to catalyze the disproportionation of superoxide into dioxygen and hydrogen peroxide. Wild type CuZnSOD and several of the mutants associated with familial amyotrophic lateral sclerosis (FALS) (Ala(4)→Val, Gly(93)→Ala, and Leu(38)→Val) were expressed in Saccharomyces cerevisiae. Purified metal-free (apoproteins) and various remetallated derivatives were analyzed by metal titrations monitored by UV-visible spectroscopy, histidine modification studies using diethylpyrocarbonate, and enzymatic activity measurements using pulse radiolysis. From these studies it was concluded that the FALS mutant CuZnSOD apoproteins, in direct contrast to the human wild type apoprotein, have lost their ability to partition and bind copper and zinc ions in their proper locations in vitro. Similar studies of the wild type and FALS mutant CuZnSOD holoenzymes in the “as isolated” metallation state showed abnormally low copper-to-zinc ratios, although all of the copper acquired was located at the native copper binding sites. Thus, the copper ions are properly directed to their native binding sites in vivo, presumably as a result of the action of the yeast copper chaperone Lys7p (yeast CCS). The loss of metal ion binding specificity of FALS mutant CuznSODs in vitro may be related to their role in ALS.

Parkinson's Disease:

Pall et al. Lancet 1987;2(8553):238-41 have shown that copper levels were significantly higher in the cerebrospinal fluid of patients with idiopathic Parkinson's disease than in the control group. Although the specific reason for elevated copper levels was not known, copper is generally high when there is chronic inflammation, such as that caused by autoimmune or undetected allergy reactions. Furthermore, a copper enzyme is required to convert tyrosine into levodopa. Therefore, elevated levels of brain copper may be an attempt to stimulate production of levodopa. However, high copper levels in the presence of antioxidant deficiencies tend to cause increased free-radical damage to nerve cell DNA.

Dexter DT et al. Brain 1991;114:1953-75. have shown alterations in the levels of iron, ferritin and other trace metals in Parkinson's disease and other neurodegenerative diseases affecting the basal ganglia. Individuals suffering from Parkinson's disease have shown both decreased and increased levels of zinc and copper. Uitti RJ et al. Can J Neurol Sci 1989;16:310-4. has found differences in regional metal concentrations in Parkinson's disease, other chronic neurological diseases, and control brains. More specifically, Forsleff L, et al. in their work in J Altern Complement Med 1999;5:57-64. found functional zinc deficiency in Parkinson's disease. Both zinc and copper function in the antioxidant enzyme superoxide dismutase (SOD). SOD tends to be low in the area of the brain involved in Parkinson's disease. In theory, therefore, low levels of zinc and copper could leave the brain susceptible to free radical damage. However, copper and zinc, as well as iron, taken in excess can also act as pro-oxidants, and all have been associated with an increased risk of developing Parkinson's disease in preliminary research. Insufficient evidence currently exists for either recommending or avoiding supplementation with zinc and copper.

Detection of Copper Abnormalities:

Imaging technologies including CT scan, PET scan, Magnetic resonance imaging (MRI), ultrasound and nuclear medicine of various organs have been used to detect disease in patients exhibiting copper abnormalities.

The diagnosis of copper abnormalities is typically made by a blood test for serum ceruloplasmin, a liver biopsy for measurement of liver copper content and a test for 24 hour urinary copper-excretion levels.

Analytical methods for measuring copper in biological and environmental samples include flame atomic absorption spectrometry, anodic stripping voltammetry, graphite furnace atomic absorption, inductively coupled plasma-atomic emission spectroscopy and inductively coupled plasma-mass spectrometry.

Detection of copper in biological samples for purposes of disease diagnosis is complicated by the fact that copper exists in a variety of discrete and separate pools. In plasma, copper is bound to ceruloplasmin (Log K=˜12), albumin (Log K=˜7), transcuprein (Log K=<7), amino acids (Log K=7 to 5) and to a lesser extent other ligands. In addition, a very small percentage of copper exists free in solution.

Diagnosis of copper-dependent pathologies depends on the distribution of copper within these pools. As such copper can be described as “good” and “bad” in terms of its ability to affect one's health status. “Good” copper is tightly bound to structures in which the Log K is 12 or greater. “Bad” copper is loosely bound to structures in which the Log K is 7 or less. Copper is considered “bad” when it is loosely bound because it is available to participate in free radical reactions that have a potentially detrimental health effect.

Total copper in biological samples is typically detected using flame atomic absorption spectrometry in which discernment between pools of copper is not possible. Alternatively, methods which resolve serum into protein and non-protein fractions have been employed. Unfortunately these methods do not discriminate between copper bound to ceruloplasmin, albumin and transcuprein. In this case, protein-bound copper still contain both good and bad copper and the usefulness of this measurement falls into question.

### BRIEF DESCRIPTION OF THE INVENTION

The present invention in one aspect provides a simple, low cost system for measuring free or loosely bound levels of copper and other metals in bodily fluids such as blood, plasma, serum, saliva, urine, tears, cerebrospinal fluid (CSF), and sweat. More particularly, the present invention utilizes enzymes such as apo-enzymes that are copper dependent for providing a solution for measuring loosely bound copper in the body. The reconstitution of apo-enzyme and its subsequent activity will be dependent upon the concentration of loosely bound copper in solution.

In one aspect of the present invention, free copper is measured in solution based on the reconstitution of apo-enzymes by copper as a cofactor. The present invention utilizes copper dependent enzymes for measurement. Soluble or immobilized apo-enzyme is exposed to copper in solution. Following exposure, copper combines with apo-enzyme forming holo-enzyme. In holo-form, the enzyme is active and capable of catalyzing a substrate. Substrate is provided in excess so that measurement of activity is a function of holo-enzyme reconstitution following combination with copper. Since the concentration of enzyme is fixed, reconstitution of holo-enzyme follows pseudo first order kinetics and is dependent on the concentration of copper in solution. Therefore the activity of enzyme measured will be a function of the concentration of copper in solution.

Copper dependent enzymes are oxidoreductases and as such activity can be measured in a variety of ways based on different analytes. Enzyme activity can be based on the formation of a product, the reduction of a redox probe or the formation of hydrogen peroxide. Measurement of product, redox probe or hydrogen peroxide can involve detection schemes that utilize optical, e.g., spectrophotometric, electrochemical or chemoluminescence technologies.

Utilization of apo-enzyme can proceed in soluble or solid state form. In soluble form, detection of analyte will depend on properties within a solution. An example of detection in solution phase would involve the use of fluorescence to detect a fluorometric product. In solid state form, detection will depend on perturbation of a surface. An example of detection in solid phase would involve the immobilization of apo-enzyme on an electrochemical strip connected to a reader. In this case, reconstitution of holo-enzyme by copper in the presence of substrate and electrochemical mediator would result in the outward flow of electrodes at a given oxidation potential.

In one embodiment, the invention permits discerning between free and bound copper in a solution such as blood. In this case, if the molar quantity of apo-enzyme is less than that of free copper in solution, then holo-enzyme will be reconstituted from the free copper pool. This also assumes that the rate constant for binding of free copper to an apo-enzyme is significantly greater that the rate constant for replacing vacated free copper from bound copper. In the case of blood, this is true. That is to say for many copper dependent enzymes, the Kd for copper binding is typically in the order of 10E-12. And for copper bound to ceruloplasmin, the Kd is also of the order of 10E-12. Therefore, the initial rate of copper binding to apo-enzyme will be a function of free copper and not bound copper.

Application of the present invention may be suited to a number of diseases in which measurement of free copper in serum can be diagnostic. Most notable is Wilson's disease. Wilson's is a genetic disease in which pathologic copper accumulation affects the health and function of the liver. In Wilson's, total serum copper can be normal or slightly decreased from normal. Total serum copper falls into pools that are bound or free. Most of the bound copper is associated with ceruloplasmin which in normal subjects represents about 80% of total copper. The free copper pool is distributed between albumin, peptides and amino acids. In Wilson's, ceruloplasmin can be decreased but it is the free copper that is increased and believed to play a diagnostic as well as a pathologic role.

### DETAILED DESCRIPTION OF THE DRAWINGS AND PREFERRED EMBODIMENTS

Further features and advantages of the present invention will be seen by the following detailed description taken in connection with the accompanying drawings, wherein like numerals depict like parts, and wherein:

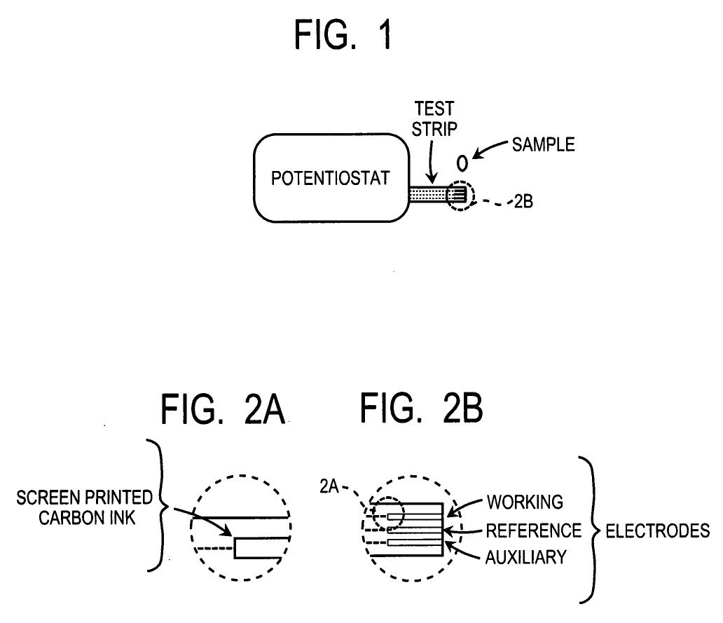

FIG. 1 is a schematic showing the relationship of a potentiostat to an electrode used to measure free copper in accordance with a preferred embodiment of the present invention;

FIGS. 2A and 2B are enlargements showing details of the electrode of FIG. 1;

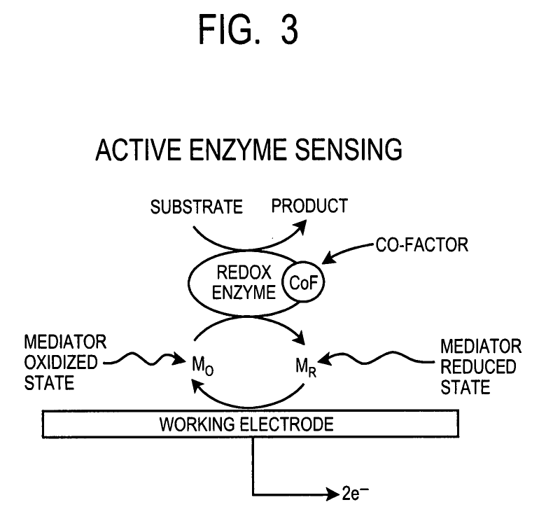

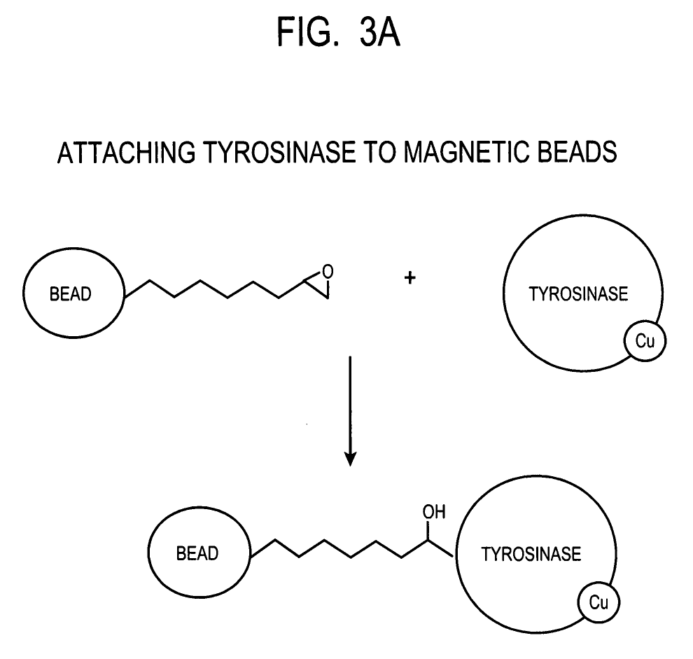

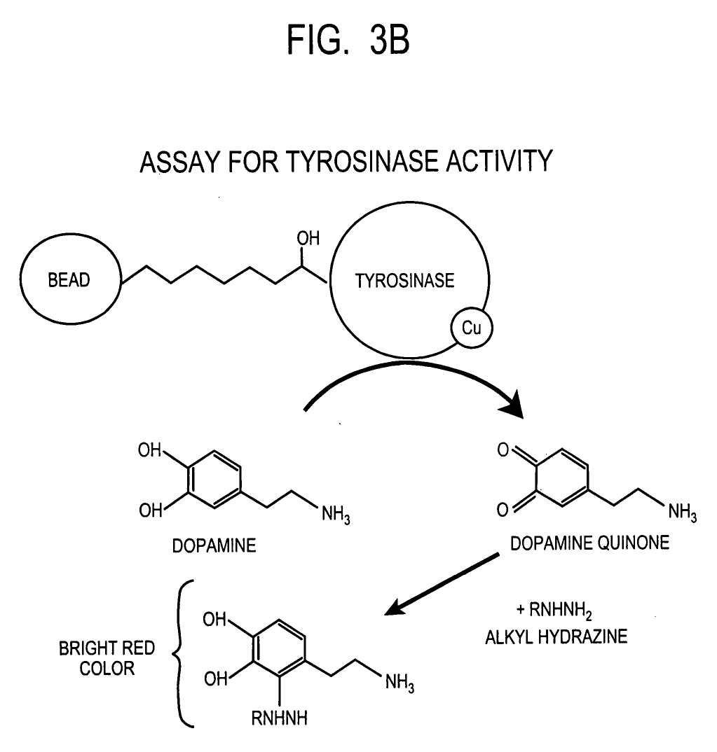

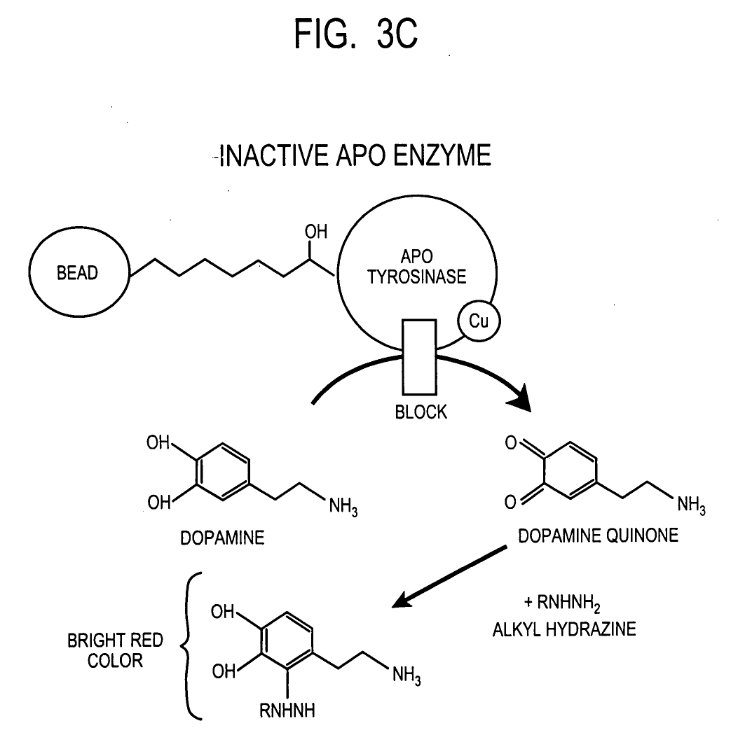

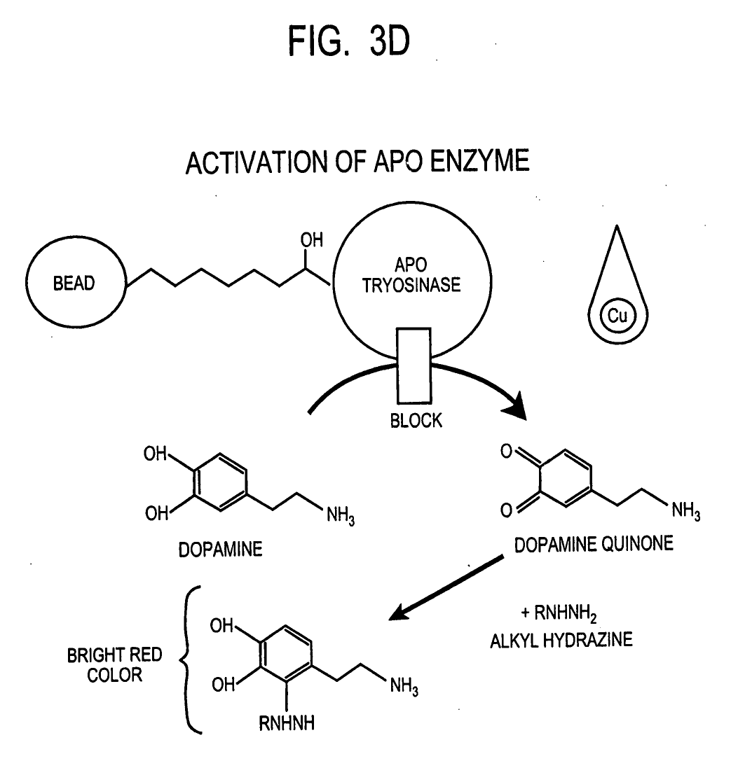

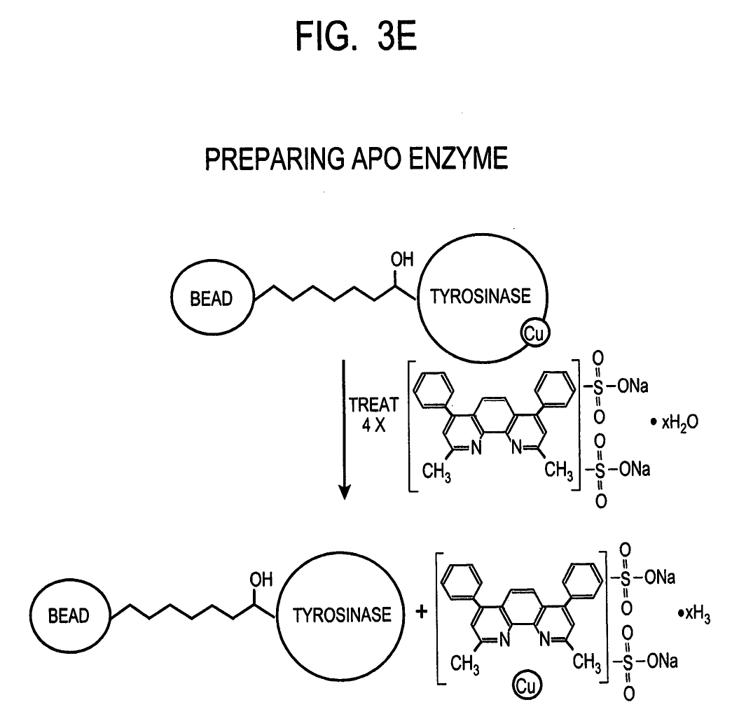

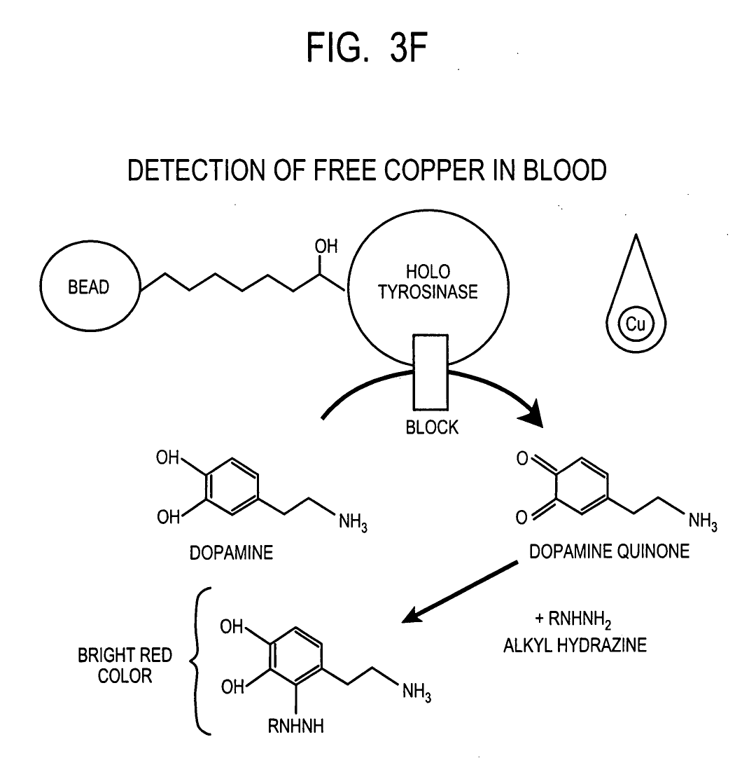

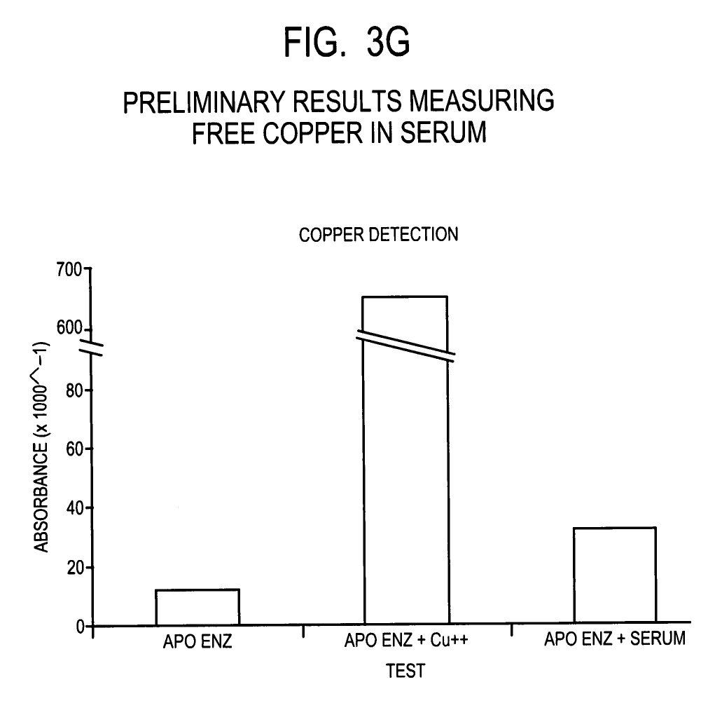

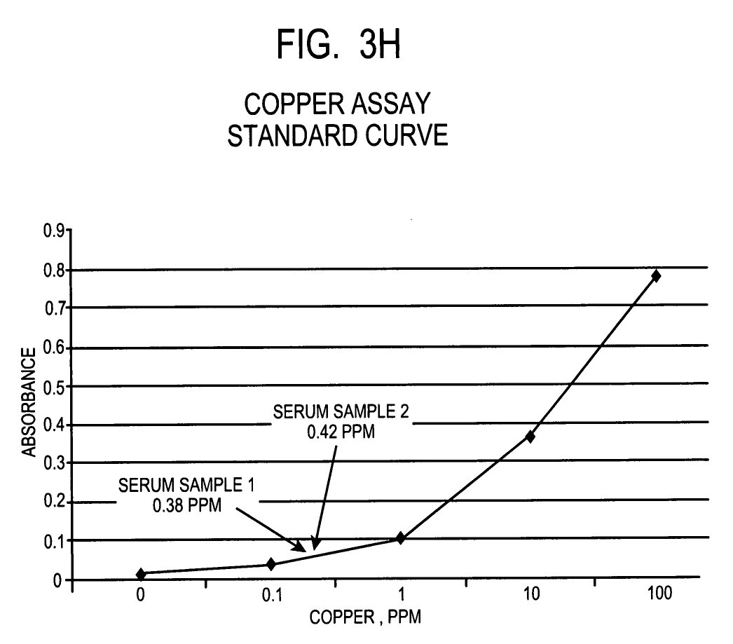

FIG. 3 is a generalized schematic for an enzymatic reaction that provides an electrochemical signal in accordance with the present invention;

FIGS. 3A-3F illustrate preparation and use of enzymatic assays in accordance with the present invention and FIGS. 3G-3H illustrate assay results;

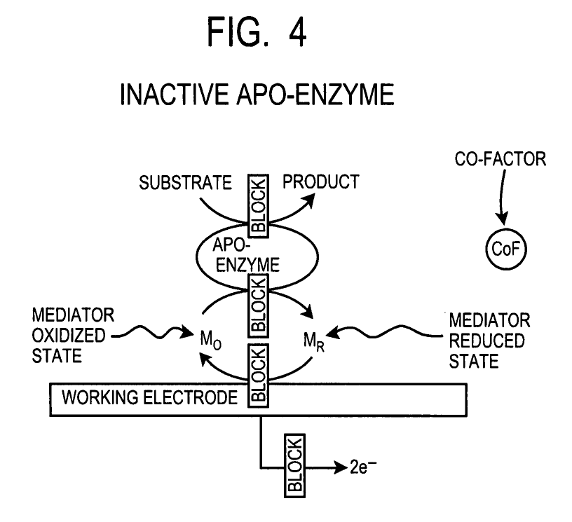

FIG. 4 is a view similar to FIG. 3, showing the effect of an enzymatic reaction in which the co-factor has been removed from the system;

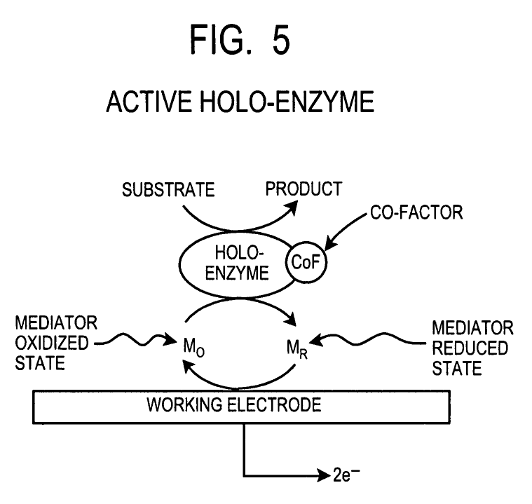

FIG. 5 is a view similar to FIG. 3, showing the effect of an enzymatic reaction in which the co-factor has been added and is properly bound to the enzyme;

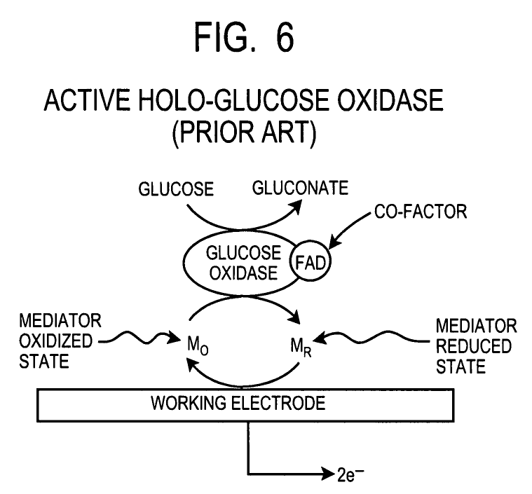

FIG. 6 is a view similar to FIG. 3, showing glucose being oxidized to gluconate by the holo-enzyme glucose oxidase, in accordance with the prior art;

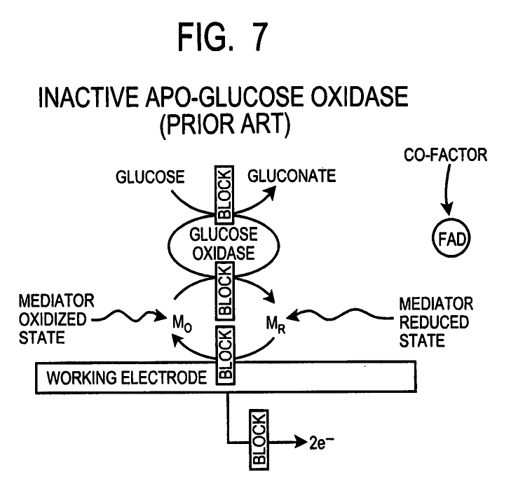

FIG. 7 is a view similar to FIG. 3, showing the loss of glucose oxidase activity when the specific co-factor FAD is removed, in accordance with the prior art;

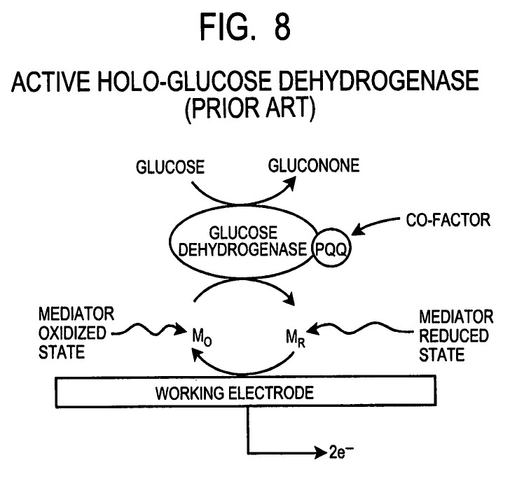

FIG. 8 is a view similar to FIG. 3, showing glucose being oxidized to gluconone by the holo-enzyme glucose dehydrogenase, in accordance with the prior art;

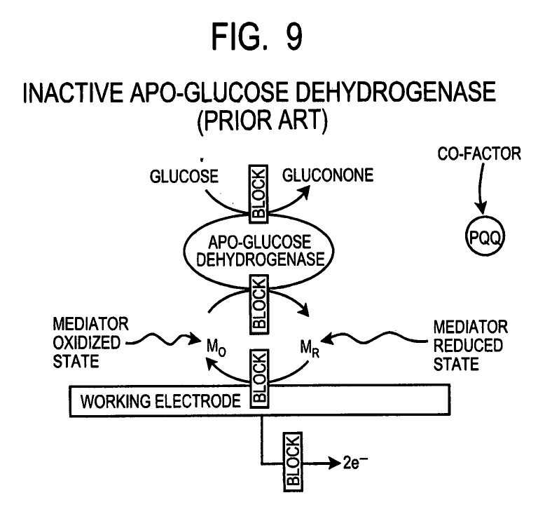

FIG. 9 is a view similar to FIG. 3, showing the loss of glucose dehydrogenase activity when the specific co-factor PQQ is removed, in accordance with the prior art;

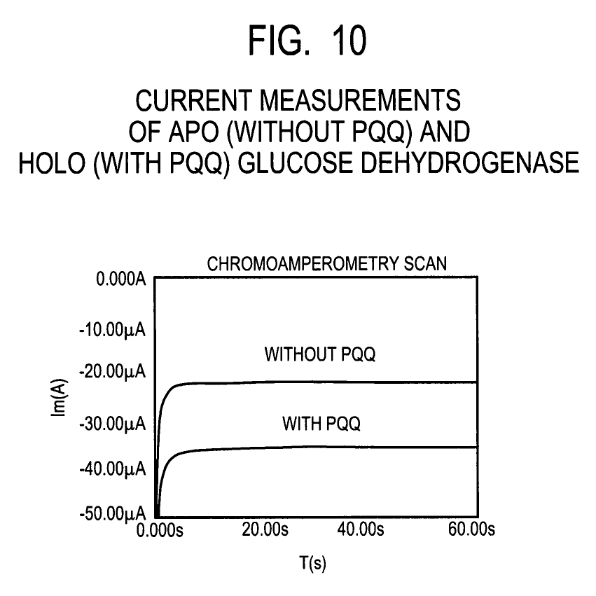

FIG. 10 are plots showing outward current (more negative) results from the addition of the co-factor PQQ (With PQQ) to glucose dehydrogenase;

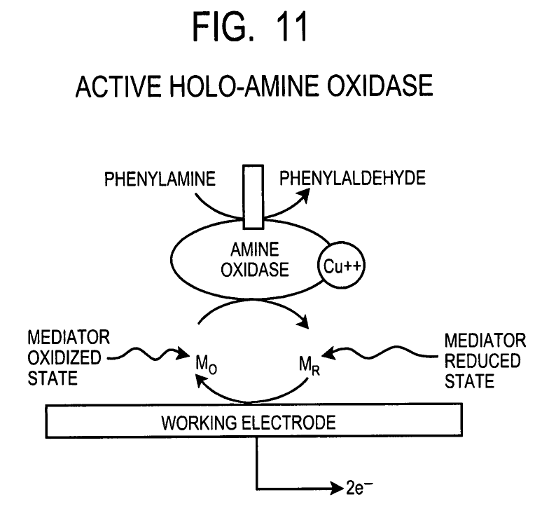

FIG. 11 is a view similar to FIG. 3, showing the action of holo-amine oxidase;

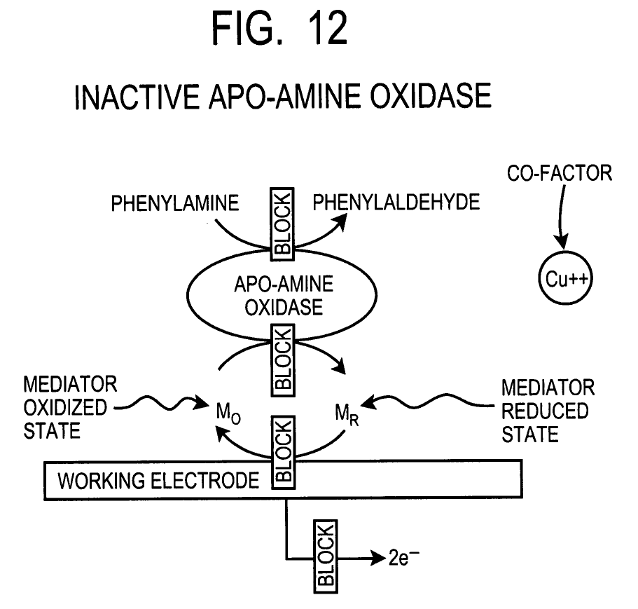

FIG. 12 is a view similar to FIG. 3, showing the inaction of apo-amine oxidase;

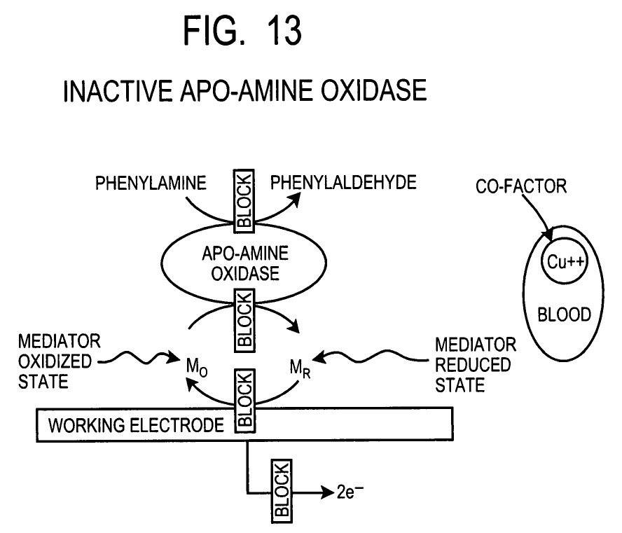

FIG. 13 is a view similar to FIG. 3, showing the inaction of apo-amine oxidase;

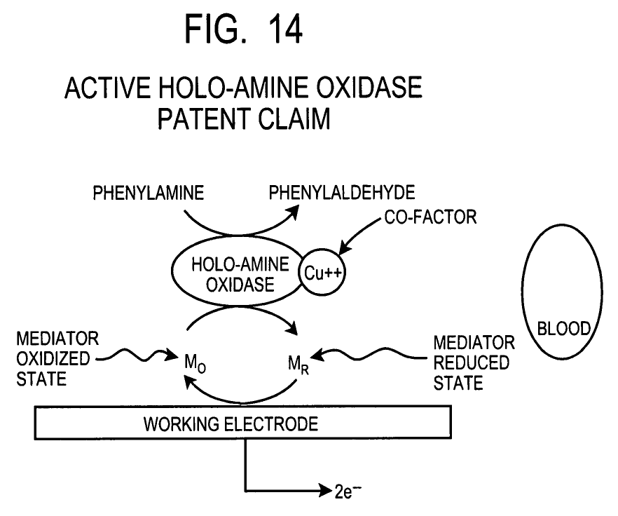

FIG. 14 is a view similar to FIG. 3, showing the activation of amine oxidase in the presence of a drop of blood;

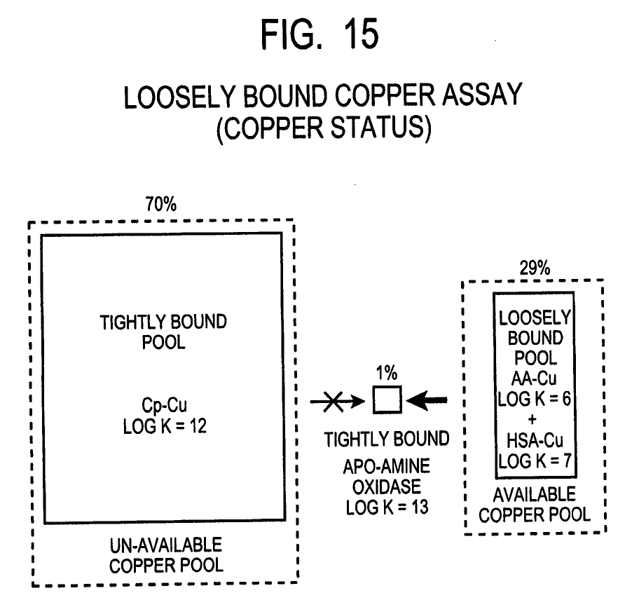

FIG. 15 is a schematic showing distribution of copper as bound to biochemical entities that are contained in a drop of blood, and the interaction with apo-amine oxidase;

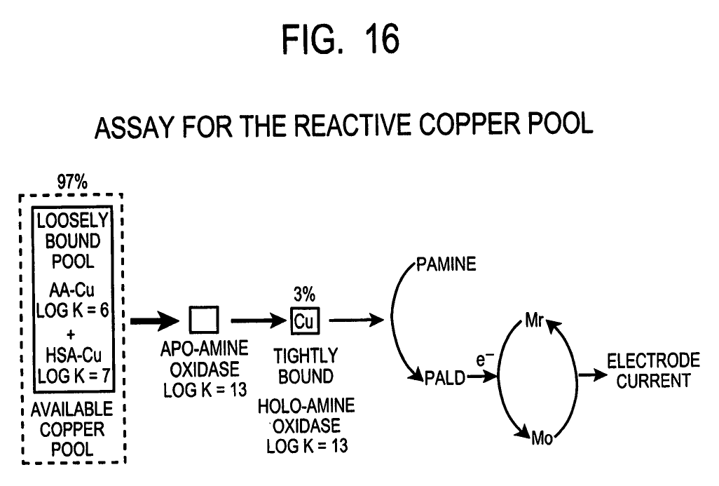

FIG. 16 is a schematic showing the interaction of the available pool of copper in a drop of blood and the interaction with apo-amine oxidase, and showing distribution of copper between the unavailable and available pools;

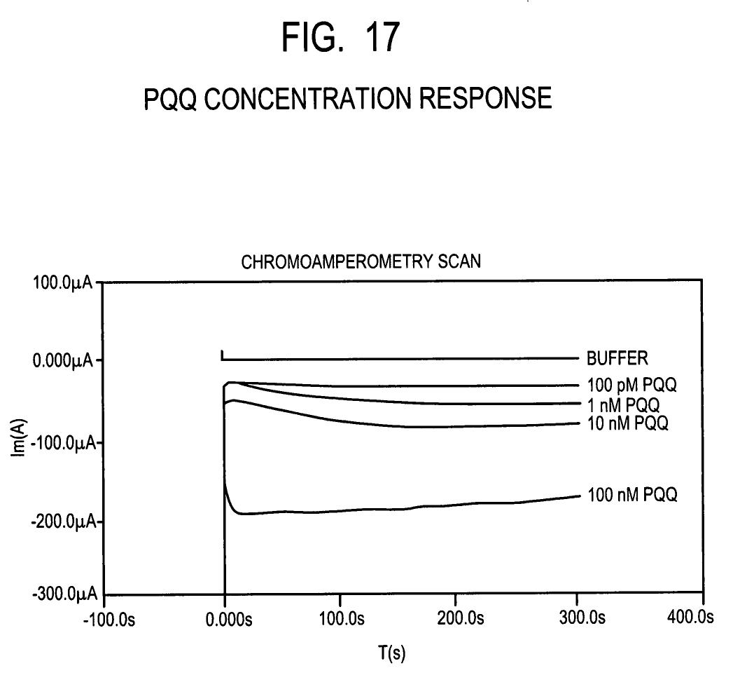

FIG. 17 are plots showing the activation of apo-glucose dehydrogenase with the addition of the co-factor PQQ in doses ranging from 100 pM to 100 Nm;

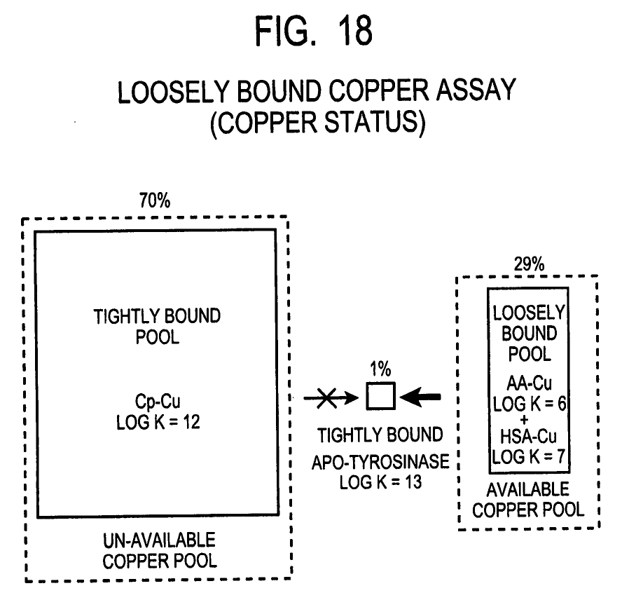

FIG. 18 is a view similar to FIG. 15 showing distribution of copper as bound to biochemical entities that are contained in a drop of blood, and the interaction with apo-tyrosinase; and

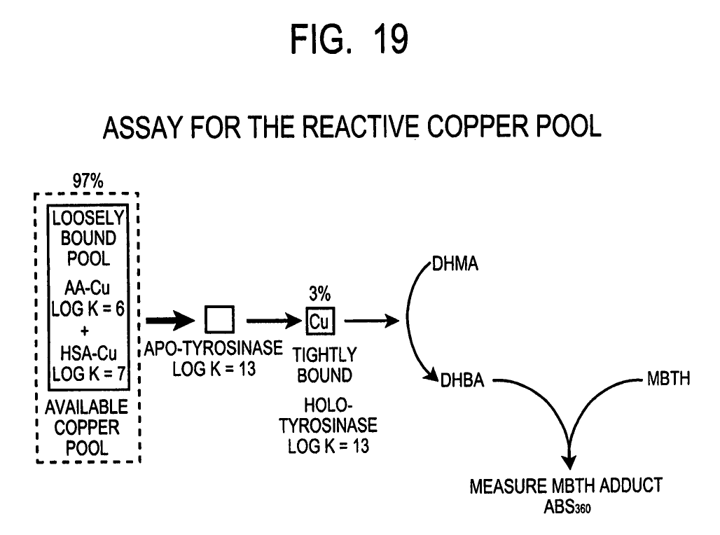

FIG. 19 is a view similar to FIG. 16 showing interaction of the available pool of copper in a drop of blood and the interaction with apo-tyrosinase, and showing distribution of copper between the unavailable and available pools.

### DETAILED DESCRIPTION OF PREFERRED EMBODIMENTS

Squitti and researchers have demonstrated that “Free Copper” tracks with the Mini Mental Status Exam (MMSE) in Alzheimer's Disease. Historically Free Copper has been measured using methods that are less than direct.

To test the hypothesis that an apo-enzyme can be used to measure free copper in a blood sample, we performed the following experiment. Magnetic beads derivatized with epoxide were mixed with Tyrosinase which resulted in the coupling of Tyrosinase to the magnetic bead. apo-Tyrosinase coupled to magnetic beads was prepared using incubation and washing four times with the copper chelator sulfobathocuprione. apo-Tyrosinase magnetic beads were then incubated with serum an then washed extensively. In this way, apo-enzyme was converted to holo-enzyme from the free copper dissolved in serum. The amount of free copper dissolved in serum will be proportional to the amount of holo-enzyme generated. A standard curved was then generated based on a colorimetric assay of holo-enzyme activity and added copper sulfate (free copper). Normal serum samples were then tested for activation of holo-enzyme using apo-Tyrosinase magnetic beads. Results gave 0.38 and 0.42 PPM for the two samples. Total copper for the two samples measured by atomic adsorption was greater than 1.2 PPM. In the same way that Tyrosinase can be attached to magnetic beads, it can also be attached to an electrode surface and holo-enzyme activity can be measured electrochemically.

FIG. 3 is a generalized schematic for an enzymatic reaction that provides an electrochemical signal. FIGS. 3A-3F illustrate preparation and use of enzymatic assays. FIG. 3G and 3H illustrate assay results. In this case an oxidase oxidezes a substrate to a product. In so doing, electrodes from the oxidation are transduced by a potentiostate connected to electrodes placed in direct contact with the reaction. The electrodes are transduced through the action of a mediator. The enzymatic reaction has an absolute requirement for the proper enzymatic binding of a co-factor.

FIG. 4 is a generalized schematic showing the effect of an enzymatic reaction in which the co-factor has been removed from the system. An apo-enzyme is an enzyme in which the co-factor has been removed. Removing the co-factor from the system causes the enzyme to be inactive. Associated enzymatic activity is therefore blocked.

FIG. 9 is a generalized schematic showing the effect of an enzymatic reaction in which the co-factor has been added and is properly bound to the enzyme. A holo-enzyme is an enzyme in which the co-factor is bound. A holo-enzyme is active and fully capable of producing a product from a substrate. As such, transduction of electrons by a working electrode is also possible.

FIGS. 6-9 are provided for comparison purposes and illustrate the prior art.

FIG. 6 is a schematic showing a specific example of glucose being oxidized to gluconate by the holo-enzyme glucose oxidase. In this case the co-enzyme is FAD and a common mediator used to transducer electrical current through a working electrode is ferrocene. This type of configuration is typical of many common glucometers currently in use.

FIG. 7 is a schematic showing the loss of glucose oxidase activity when the specific co-factor FAD is removed. As an apo-enzyme, glucose oxidase is not capable of oxidizing glucose. As a result, transduction of current is blocked.

FIG. 8 is a schematic showing a specific example of glucose being oxidized to gluconone by the holo-enzyme glucose dehydrogenase. In this case the co-enzyme is PQQ and a common mediator used to transducer electrical current through a working electrode is ferrocene. This is another type of configuration in glucometers today.

FIG. 9 is a schematic showing the loss of glucose dehydrogenase activity when the specific co-factor PQQ is removed. As an apo-enzyme, glucose oxidase is not capable of oxidizing glucose. As a result, transduction of current is blocked.

FIG. 10 shows data in which outward current (more negative) results from the addition of the co-factor PQQ (With PQQ) to glucose dehydrogenase.

FIG. 11 shows the action of holo-amine oxidase. In this case the substrate phenylamine is oxidized to the product phenylaldehyde. Enzyme activity requires the proper binding of copper as a co-factor to amine oxidase. Outward current is transduced through a chemical medicator at the surface of a working electrode.

FIG. 12 shows the inaction of apo-amine oxidase. In this case the substrate phenylamine is not oxidized to phenylaldehyde because the co-factor copper has been removed resulting in the formation of apo-amine oxidase. As a result, outward current is not transduced through a chemical mediator at the surface of a working electrode.

FIG. 13 shows the inaction of apo-amine oxidase. In this case the substrate phenylamine is not oxidized to phenylaldehyde because the co-factor copper has been removed resulting in the formation of apo-amine oxidase. As a result, outward current is not transduced through a chemical mediator at the surface of a working electrode. This system is configured in preparation for the introduction of a drop of blood that contains the co-factor copper.

FIG. 14 shows the activation of amine oxidase in the presence of a drop of blood. In the presence of a drop of blood, the co-factor copper is delivered to a binding site on the enzyme which restores activity and allows for current to be measured. The amount of current that is measured is proportional to the concentration of free copper in the drop of blood.

FIG. 15 shows distribution of copper as bound to biochemical entities that are contained in a drop of blood. The major entities include ceruloplasmin (Cp-Cu), human serum albumin (HSA-Cu) and amino acids (AA-Cu). When a drop of blood is applied to the test strip, the apo-amine oxidase printed on the working electrode would represent an addition 1% of copper binding. Apo-amine oxidase has a large binding constant and would extract copper from the loosely bound pool. At only 1% binding, apo-amine oxidase would not significantly perturb this copper status especially regarding the tightly bound pool. As a result, the activity of apo-amine oxidase reconstitution only would be determined by the concentration of copper in the loosely bound pool.

FIG. 16 shows the interaction of the available pool of copper in a drop of blood and the interaction with apo-amine oxidase. In FIG. 15, the distribution of copper is shown between the unavailable and available pools. However, in this FIG. 16 the distribution of copper is shown between the available pool and holo-amine oxidase. The total distribution of copper in this situation is defined as 100% and once again we see that the impact of apo-amine oxidase is negligible on this pool. Therefore the rate of activation of apo-amine oxidase which in turn determines the amount of electrode current, will be a function of the concentration of copper in the loosely bound pool. The enzyme activity of the holo-amine oxidase involves the oxidation of phenylamine (PAMINE) to phenylaldehyde (PALD). This results in the outward flow of electrons which are transduced to the surface of an electrode by a chemical mediator (Mr→Mo).

FIG. 17 shows data illustrating the activation of apo-glucose dehydrogenase with the addition of the co-factor PQQ. PQQ was added to apo-enzyme in doses ranging from 100 pM to 100 nM. The addition of PQQ resulted in an increase in the measurement of outward current (more negative amps). This demonstrates the principle of an apo-enzyme with capability of distinguishing between increasing concentrations of added co-factor. In this case, distinctions were observed by 60 sec.

FIG. 18 shows distribution of copper as bound to biochemical entities that are contained in a drop of blood. The major entities include ceruloplasmin (Cp-Cu), human serum albumin (HSA-Cu) and amino acids (AA-Cu). For optical detection, plasma or serum is isolated. When the plasma is mixed with reagents including enzyme for optical detection, the apo-tyrosinase would represent an addition 1% of copper binding. Apo-tyrosinase has a large binding constant and would extract copper from the loosely bound pond. At only 1% binding, apo-tyrosinase would not significantly perturb this copper status especially regarding the tightly bound pool. As a result, the activity of apo-tyrosinase reconstitution only would be determined by the concentration of copper in the loosely bound pool.

FIG. 19 shows the interaction of the available pool of copper in a drop of blood and the interaction with apo-tyrosinase. In FIG. 23, the distribution of copper is shown between the unavailable and available pools. However, in this FIG. 24 the distribution of copper is shown between the available pool and holo-tyrosinase. The total distribution of copper in this situation is defined as 100% and once again we see that the impact of apo-tyrosinase is negligible on this pool. Therefore the rate of activation of apo-tyrosinase which in turn determines the amount of product formed, will be a function of the concentration of copper in the loosely bound pool. The enzyme activity of holo-tyrosinase involves the oxidative decarboxylation of 3,4-dihydroxymandelic acid (DHMA) to 3,4-dihydroxybenzaldehyde (DHBA). In turn, DHBA reacts with 3-methyl-2-benzothiazolinone hydrazone (MBTH) to form an adduct with a large extinction coefficient. Absorption of the MBTH adduct is then measured at 360 nm.

Accordingly, since the method of the present invention is more convenient than the current conventional methods of diagnostic testing (i.e. flame atomic absorption spectrometry) the specimen required for testing, it is now possible that a point of care diagnostic of Wilson's disease can be achieved with the current method.

While the invention has been shown and described with reference to certain preferred embodiments thereof, it will be understood by those skilled in the art that various changes in form and details may be made therein without departing from the spirit and scope of the invention as defined by the appended claims

What is claimed is:

1. A detection method or a method for a point of care diagnostic assay which comprises detecting non-covalently bound copper (free copper) concentrations in a fluid.

2. The method of claim 1, wherein the fluid comprises a bodily fluid selected from the group consisting of blood, plasma, serum, saliva, urine, tears, cerebrospinal fluid and sweat.

3. The method of claim 1, wherein the free copper is measured by electrochemical detection, preferably using a two or a three electrode electrochemical detection system.

4. A method of claim 3, wherein the electrode system comprises three electrodes, a working electrode, a reference electrode, and a counter or auxiliary electrode, or a working electrode, and a combined reference and counter or auxiliary electrode.

5. The method of claim 3, wherein at least one of the electrodes is formed of a material selected from the group consisting of carbon, gold, silver, silver chloride, platinum and palladium.

6. A method of diagnosing Alzheimer's disease, Parkinson's disease, schizophrenia, Wilson's disease or other copper metabolic disorder in an individual, which comprises directly measuring a free copper pool in a fluid sample of that individual.

7. An assay device for detection of free or loosely bound copper comprising an enzyme capable of forming a holo-enzyme in the presence of copper.

8. An assay device as claimed in claim 7, wherein the holo-enzyme comprises an active enzyme, preferably an active apo-enzyme, or an inactive apo-enzyme.

9. An assay device for detection of free or loosely bound copper, which comprises an enzyme which uses copper as a co-factor.

10. The assay device as claimed in claim 9, wherein the enzyme comprises amine oxidase.

11. The assay device as claimed in claim 8, wherein (a) apo-enzyme is prepared by removing copper, or (b) apo-enzyme is inactive in the absence of copper, or (c) when a sample containing copper is added to an apo-enzyme, activity is restored; or (d) wherein when copper is added to an apo-enzyme to form a holo-enzyme, activity is restored, or (e) wherein activity of a holo-enzyme is measured.

12. An assay device for detection of copper, wherein enzyme activity is measured using a spectrophotometric or electrochemical system.

13. The assay device as claimed in claim 12, wherein, in the case of spectrophotometric system, changes in optical properties of a substrate or an electrochemical mediator are measured, and in the case of an electrochemical system, changes in electrochemical properties of a solution are measured.

14. The assay device as claimed in claim 12, wherein electrochemical changes in a solution are measured electrochemically in the presence of a chemical mediator.

15. The assay device as claimed in claim 14, wherein a chemical mediator is used to transduce electrochemical changes to an electrode.

16. The assay device as claimed in claim 15, wherein said electrochemical changes include an increase or a decrease in current flow when a fixed potential is applied.

17. The assay device as claimed in claim 10, wherein the amine oxidase is oxidized whereby to generate a flow of electrons from the amine substrate; the electrons are captured by a chemical mediator that exists in an oxidized state, whereby the mediator is chemically transformed into a reduced state; and the chemical mediator is then oxidized at the surface of an electrode, wherein facilitation of the oxidation of the reduced chemical mediator occurs when a fixed potential is applied to the electrode.

18. The assay device as claimed in claim 12, wherein a two electrode system is used to detect enzyme activity, in which a working electrode acts as a catalyst for the oxidation of a chemical mediator, and an auxiliary electrode acts as a counter electrode and as a reference electrode for a flow of electrons, wherein in the case of oxidation of the chemical mediator the flow of electrons is inward.

19. The assay device as claimed in claim 12, wherein a three electrode system is used to detect enzyme activity, in which a working electrode acts a catalyst for the oxidation of the chemical mediator, an auxiliary electrode acts to complete a circuit and to provide a flow of electrons, and a reference electrode provides feedback and establishes an absolute potential depending on a chemical composition of the reference electrode.

20. The assay device as claimed in claim 12, wherein an electrode is connected to a potentiostat which provides for electrochemical measurements, the potentiostat is programmed to establish a fixed potential in a range for oxidizing an enzymatically reduced mediator, wherein at a fixed potential setting, a current that results from oxidation of the chemical mediator is recorded as a change in current above background.

21. The assay device as claimed in claim 12, wherein a change in current provides a measure of enzyme activity, in which enzyme activity is a direct measure of the free copper concentration which converts inactive apo-enzyme into active holo-enzyme.

22. The assay device as claimed in claim 19, wherein calibration of current vs. free copper in solution at various concentrations provides utility regarding a single-point measurement of the concentration of free copper in a test solution.

23. The assay device as claimed in claim 19, wherein the potentiostat is interfaced to a display for providing a reading indicating a concentration of free copper in a solution including blood.

24. The assay device as claimed in claim 19, wherein the electrodes are constructed so that measurement of free copper is made using solution phase or solid-state phase methods.

25. The assay device as claimed in claim 24, characterized by one or more of the following features:
(a) wherein in the case of a solution phase method, the working electrode is used in an unmodified state, and all of the reagents exit in a solution phase;
(b) wherein in the case of a solid phase method, the working electrode is modified so that one or more reagents exit with the working electrode in a solid state;
(c) wherein the enzyme and/or the chemical mediator are mixed with materials forming the working electrode;
(d) wherein the working electrode is screen printed with conductive ink that is doped with enzyme and/or chemical mediator;
(e) wherein a surface of the working electrode is screen printed with a conductive carbon ink doped with enzyme and/or chemical mediator and covered with a membrane that is selective for copper permeation;
(f) wherein the working electrode is screen printed with a conductive ink which also includes a conductive material, preferably carbon, gold or silver;
(g) wherein the working electrode is screen printed with conductive ink that includes a doping material selected from amine oxidase or other redox-type enzyme, or ferrocine, PMS, DCPIP or other type chemical mediator; and
(h) wherein a membrane formed of a polymerizing material that provides copper selectivity covers the working electrode at least in part, wherein the membrane preferably is formed of nafion.

26. The assay device as claimed in claim 12, wherein a two electrode system is used in which current is generated using a first working, electrode specific to copper detection and a second working electrode that is not specific to copper detection; and specific copper detection is achieved by subtracting a current generated at the non-specific electrode from the current generated at the specific electrode.

27. A device that measures the state of copper in blood as a gradient of free to loosely to tightly bound copper is measured, wherein free copper in blood is based on a measurement of enzyme activity as an initial rate and not at equilibrium.

28. The device as claimed in claim 27, wherein enzyme activity as an initial rate provides for measurement of the free form of copper in blood.

29. The device as claimed in claim 27, using the assay as claimed in claim 50(e), wherein specific measurement of the free form of copper as an initial rate is achieved due to a limited number of enzymes doped into the conductive carbon ink as compared to the total number of free copper molecules in a drop of blood.

30. The device as claimed in claim 29, characterized by one or more of the following features:
(a) wherein at 1 uM of free copper in a 20 ul sample of blood, the total number of copper molecules is 1 E+13, the total number of copper binding sites on the electrode is E+9 or less, and even if all the free copper binding sites on the electrode were occupied, the free copper concentration of copper the would not be significantly altered even for normal patients;
(b) wherein, enzyme activity is based on the most accessible, free copper concentration; and
(c) wherein initial rates of enzyme activity is measured within 1 minute to avoid saturation of enzyme sites on the electrode, and further to avoid perturbation of copper homeostasis in the measured drop of blood.

31. An assay device for detection of free or loosely bound copper comprising a copper-dependent enzyme attached to a bead.

32. The assay device of claim 31, wherein the enzyme comprises an apo-enzyme attached to a bead by removing copper.

33. A method for measuring the concentration of copper in serum or blood by reconstitution of an enzyme attached to a bead from an apo-form to a holo-form.

34. The method of claim 33, wherein activity of a reconstituted apo-enzyme from copper in serum or blood is proportional to the concentration of copper in the serum of blood.
